(19) United States
(12) Patent Application Publication
(10) Pub. No.: US 2023/0301744 A1
(43) Pub. Date: Sep. 28, 2023

(54) RESILIENT DOCK, SUCH AS A FOAM DOCK, FOR ORGANIZING MEDICAL EQUIPMENT USING FOR ENDOSCOPIC PROCEDURES

(71) Applicant: New York University, New York, NY (US)

(72) Inventor: Jovanka MONTANEZ, New York, NY (US)

(21) Appl. No.: 18/125,700

(22) Filed: Mar. 23, 2023

Related U.S. Application Data
(60) Provisional application No. 63/323,274, filed on Mar. 24, 2022.

(51) Int. Cl. A61B 90/50; A61B 50/26

(52) U.S. Cl. CPC A61B 90/50; A61B 50/26

(57) ABSTRACT

An endoscopic surgical tool organizer includes a holder section having an upper surface, a substantially flat lower surface, and at least one peripheral edge. The upper surface has a plurality of slits or channels, each having an entry point and an exit point on the at least one peripheral edge, and each dimensioned to hold at least a portion of a length of an endoscopic insertion tube or endoscopic tool wire or tube. The lower surface is provided with a non-slip material, finish, or adhesive. The holder section is dimensioned to fit on a tray of a mayo stand. In some cases, two or more of the plurality of slits or channels have substantially parallel paths, or paths serve to spread out or concentrate the wires and/or tubes.

### § 1. RELATED APPLICATION(S)

This application claims the benefit of U.S. Provisional Application No. 63/323,274 (referred to as “the ′274 provisional” and incorporated herein by reference), titled “FOAM DOCK FOR ORGANIZING MEDICAL EQUIPMENT FOR ENDOSCOPIC PROCEDURES,” filed on Mar. 24, 2022, and listing Jovanka MONTANEZ as the inventor. The scope of the invention is not limited to any requirements of the specific embodiments in the ′274 provisional.

### § 2. BACKGROUND

### § 2.1 Field of the Invention

The present invention concerns medical equipment used during medical procedures, such as during endoscopic surgery.

### § 2.2 Background Information

The organization of medical equipment (such as medical instruments, supplies, and the like) utilized during medical procedures can present various challenges. In some procedures, such as endoscopy procedures, instruments may be placed with no support on a workspace used to during medical procedures, such as a mayo stand (See, e.g., FIG. 1.) or an endoscopy tower (not shown). Referring to FIG. 1, an example mayo stand 100 may include a tiltable tray top 110 supported in a pivoting manner by a support frame 120. The support frame 120 is, in turn, supported by one or more risers 130 and one or more brackets 140. The risers 130 are, in turn, supported by a base potion (not shown).

This unsecured positioning of medical equipment on the workspace may result in the equipment falling off of the workspace and onto the floor or into an open bag hanging off of the workspace (See, e.g., one side of saddle bag 210 in FIG. 2.) during a surgical procedure. These situations often require medical support staff to retrieve and re-sterilize the displaced items for further use during the procedure. FIGS. 3A and 3B illustrate various medical equipment 310 placed on a tray 320 (e.g., of a mayo stand). As shown, some of the equipment 310, such as tubes, wires, etc., are draped over an edge of the tray 310 and into an opened bag 330, such as a saddle bag. As shown in FIG. 3C, tubes and/or wires 340 may be held in the opened bag 320′.

As should be apparent from FIGS. 3A-3C, it is difficult to maintain medical equipment 310 in an organized manner, especially when equipment is being removed from the tray 320 and/or replaced back onto the tray 320. This problem is exacerbated when the medical equipment 310 includes tubes and/or wires which may be long, coiled, partially uncoiled, cross over and/or under each other and/or other equipment, etc. Therefore, there is a need in the art for a device and technique to effectively organize equipment for use during medical procedures, and especially in the case of endoscopic surgery which often has long tubes and/or wires.

### § 3. SUMMARY OF THE INVENTION

An endoscopic surgical tool organizer consistent with the present description includes a holder section having an upper surface, a substantially flat lower surface, and at least one peripheral edge. The upper surface has a plurality of slits or channels, each having an entry point and an exit point on the at least one peripheral edge, and each dimensioned to hold at least a portion of a length of an endoscopic insertion tube or endoscopic tool wire or tube. The lower surface is provided with a non-slip material, finish, or adhesive. The holder section is dimensioned to fit on a tray of a mayo stand.

In some example embodiments of the endoscopic surgical tool organizer, the holder section is made from foam.

In some example embodiments of the endoscopic surgical tool organizer, the holder section is a foam block, or a foam disk.

In some example embodiments of the endoscopic surgical tool organizer, at least one of the plurality of slits or channels is a slit.

In some example embodiments of the endoscopic surgical tool organizer, at least one of the plurality of slits or channels is a channel having a U-shaped cross section.

In some example embodiments of the endoscopic surgical tool organizer, at least one of the plurality of slits or channels is a channel having a bulb-shaped cross section or a dovetail-shaped cross section.

In some example embodiments of the endoscopic surgical tool organizer, at least one of the plurality of slits or channels is a channel having a partially tapered cross section.

In some example embodiments of the endoscopic surgical tool organizer, at least two of the plurality of slits or channels have substantially parallel paths.

In some example embodiments of the endoscopic surgical tool organizer, at least two of the plurality of slits or channels have paths that spread out at an entry or exit side of the at least two of the plurality of slits or channels.

In some example embodiments of the endoscopic surgical tool organizer, at least two of the plurality of slits or channels have paths that concentrate at an entry or exit side of the at least two of the plurality of slits or channels.

In some example embodiments of the endoscopic surgical tool organizer, at least two of the plurality of slits or channels have paths that spread out at an entry or exit side of the at least two of the plurality of slits or channels, and that concentrate at an opposing exit or entry side of the at least two of the plurality of slits or channels.

In some example embodiments of the endoscopic surgical tool organizer, at least two of the plurality of slits or channels have paths that spread out at both an entry and an exit side of the at least two of the plurality of slits or channels.

Example methods for using the example endoscopic surgical tool organizers are also described.

A kit including a substantially circular endoscopic tool organizer and a substantially ring-shaped head rest is also described.

### § 4. BRIEF DESCRIPTION OF THE DRAWINGS

FIG. 1 illustrates a conventional mayo stand used in surgical settings.

FIG. 2 illustrates a conventional saddle bag used in surgical settings.

FIGS. 3A-3C illustrate conventional medical equipment, including wires and/or tubes, used during surgery and arranged on a conventional mayo stand having a conventional saddle bag.

FIG. 4 is a partial perspective view, and FIG. 5 is a bottom view, of an example dock consistent with the present description.

FIGS. 6A-6E are side-views of alternative channels having various alternative cross-sections.

FIGS. 7A-7D are top-plan views of alternative example square or rectangular docks.

FIG. 8-10 illustrate an example dock used in the context of endoscopic retrograde cholangiopancreatography (ERCP), endoscopic submucosal dissection (ESD) and gastrointestinal endoscopic mucosal resection (EMR), respectively, medical procedures.

FIG. 11 illustrates a kit including a dock consistent with the present description and a patient head rest.

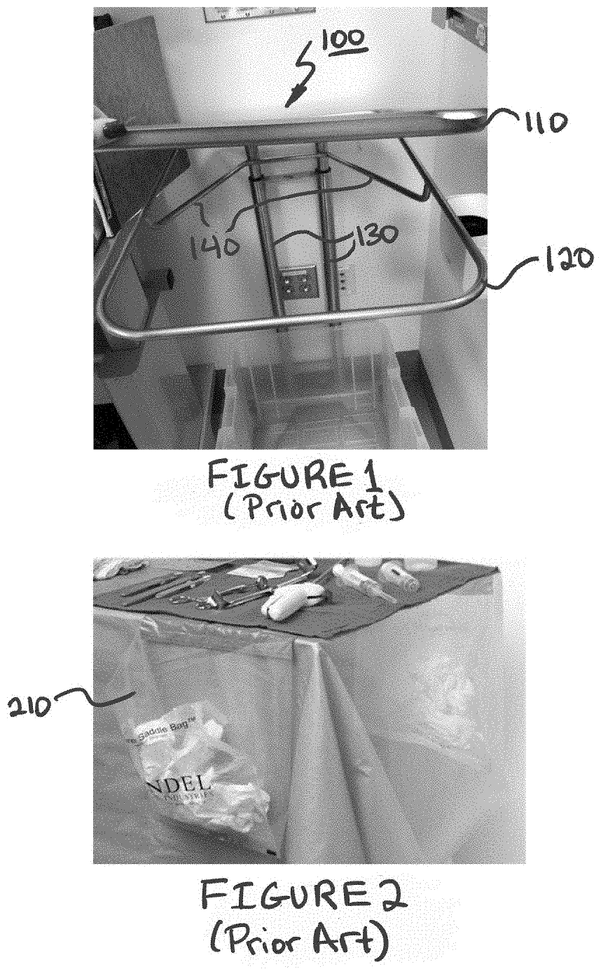

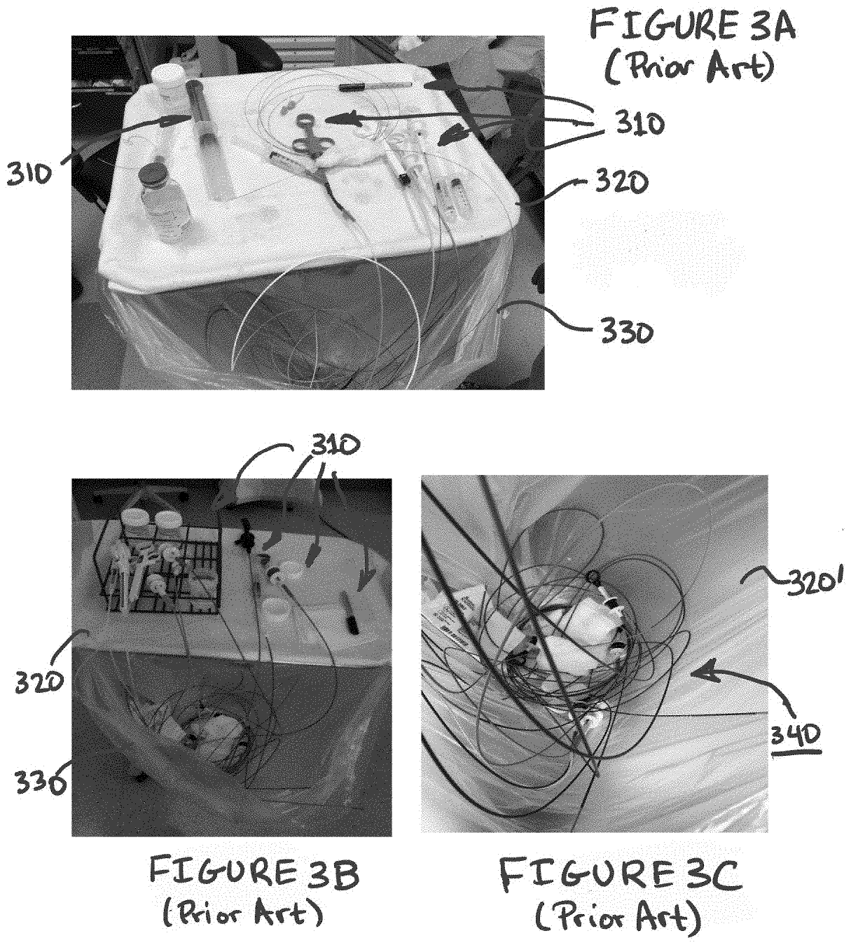

### § 5. DETAILED DESCRIPTION

Example embodiments consistent with the present description are directed to a surgical tool organizer and methods for retrievably securing medical equipment, such as medical instruments, supplies, and the like, for use during a medical procedure. In various example embodiments, the surgical tool organizer is suitably sized and shaped for secure placement on a workspace (such as a tray of a mayo stand or endoscopy tower) during a medical procedure. Note that the tray of existing mayo stands can be about 12-26 inches wide and about 19-30 inches deep (e.g., 12×19 inches, 16×21 inches, 20×25 inches, 26×30 inches, etc.). However, the example embodiments can be used with trays or surfaces having different dimensions. In one or more example embodiments, the surgical tool organizer may be rectangular parallelepiped in shape, although in other example embodiments, the surgical tool organizer may be other three-dimensional shapes (e.g., disk or fat cylindrical, cubic, irregular, etc.).

### § 5.1 Example Docks and Kits

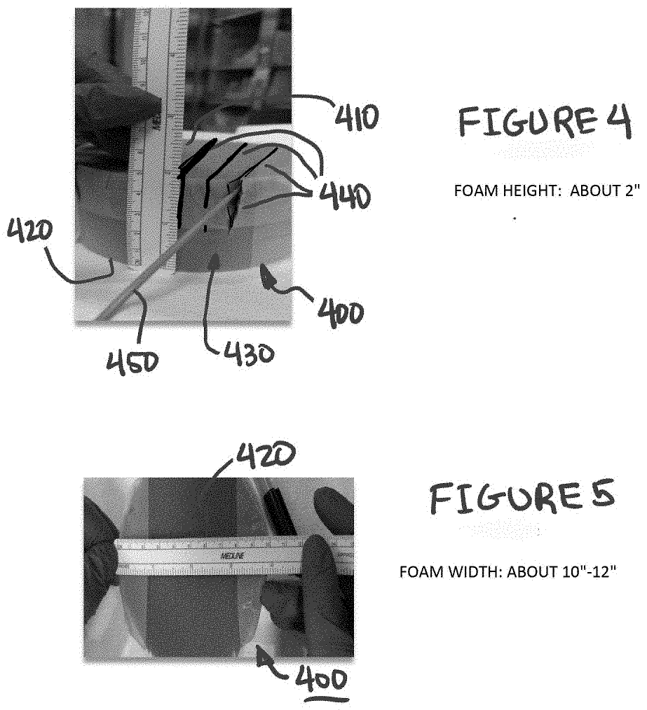

FIG. 4 is a partial perspective view of an example surgical tool organizer (also referred to as a “dock” or “holder section” without loss of generality) 400 consistent with the present description. FIG. 5 is a bottom view of the example dock 400. Referring to FIGS. 4 and 5, the surgical tool organizer 400 comprises a holder section having a (e.g., substantially flat) upper surface 410, a substantially flat lower surface 420 and a peripheral edge or edges 430. The dock 400 is shaped and dimensioned to fit on a desired workspace. For example, the dock 400 may be rectangular in shape (e.g., a block), although in other embodiments it may be a different shape such as disk or fat cylinder, or any other suitable shape. Referring back to FIG. 4, the upper surface 410 has a plurality of slits and/or channels 440, each having an entry point and an exit point (only one of which for each of three slits are shown) on the peripheral edge(s) 430, and each dimensioned to hold at least a portion of a medical instrument or supply, such as a portion of a length of an endoscopic insertion tube or endoscopic tool wire or tube 450.

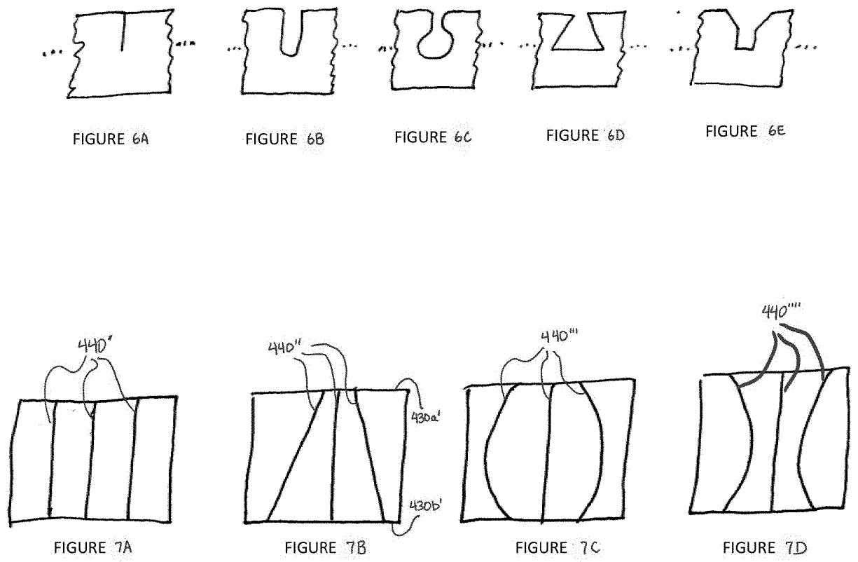

The consistency of the surgical tool organizer material along with the size and shape of the slits or channels 440 are such that the medical instruments and/or supplies are securely held within the slits or channels 440 but can be rapidly removed from and returned to (and in some example embodiments, pulled and/or pushed through) the holding section as needed. For example, the cross-section of FIG. 6A shows a slit cut into a portion of a foam dock. The cross-section of FIG. 6B shows a U-shaped channel formed in the dock. The cross-section of FIG. 6C shows a bulb-shaped channel formed in the dock. The cross-section of FIG. 6D shows a dovetail-shaped channel formed in the dock. Finally, the cross-section of FIG. 6E shows a partially tapered channel formed in the dock.

In some example embodiments, the lower surface of the holder section (Recall, e.g., 420 of FIGS. 4 and 5.) may be provided with a non-slip material, finish, or adhesive to maintain the holder section in place (e.g., on a top surface of a table or stand) during use. For example, the holder section may have on its underside 420 an adhesive strip with a removable covering that is peeled off when the surgical tool organizer 400 is ready to be removably affixed to the desired workspace. In other embodiments, other types of adhesives and/or techniques may be used for removably securing the surgical tool organizer 400 to the workspace. For example, a double-sided adhesive tape may be used to removably secure the surgical tool organizer 400 to the workspace, such as the tray of a mayo stand, or a horizonal surface of an endoscope tower.

The slits or channels of the holding section are suitably sized (length, width, and depth) and shaped (cross-sectional shape) to secure the medical equipment or supplies while allowing easy insertion and easy removal of the items as needed. In some embodiments, the slits or channels may be simple vertical cuts in the holding section (Recall, e.g., FIG. 6A). A slit or channel having a depth of from 1 to 1.5 inches has been found to be suitable for this purpose, especially if the dock is formed of foam. In certain embodiments, the slits or channels may have substantially U-shaped cross-sections. (Recall, e.g., FIG. 6B.) Additionally, or alternatively, the channels may have one or more inward-facing protrusions to hold the inserted medical instrument or supply in place. (Recall, e.g., the upper portions of FIGS. 6C and 6D.) The slits or channels may extend the width of the holding section, for example on the order of 10 inches (See, e.g., FIG. 5.), each having an entry point and an exit point on the peripheral edges of the holding section.

FIGS. 7A-7D are plan views of the upper surfaces (Recall, e.g., 410.) of alternative example square or rectangular docks. Referring first to FIGS. 7A and 7B, the slits or channels may be substantially linear along the width of the holding section. More specifically, in FIG. 7A, the slits or channels 440′ are substantially parallel with one another. On the other hand, in FIG. 7B, the linear slits or channels 440″ are concentrated on one side 430a′ and spread on the opposing side 430b′. Such an arrangement may be useful when a wire or tube is connected to a wider part of the equipment. The portions of equipment with the wider parts may be located on the spread side 430b′. Referring next to FIGS. 7C and 7D, in other embodiments, at least some of the slits or channels 440′″ and 440″, respectively, as viewed from above, may be curved (e.g., C-shaped, S-shaped, or the like). More specifically, FIG. 7C illustrates an example embodiment having a straight slit or channel and two C-shaped slits or channels which concentrate the tube(s) and/or wire(s) on both sides. FIG. 7D illustrates an example embodiment having a straight slit or channel and two C-shaped slits or channels which spread the tube(s) and/or wire(s) on both sides. Although not shown, S-shaped (and reverse S-shaped) slits or channels allow the tube(s) and/or wire(s) to be concentrated on one side and spread on the opposing side. The ends of the slits or channels (that is, the entry/exit points of the slits or channels) may be any suitable shapes (e.g., at edges of circle, square, or rectangle (or other shape). To reiterate, the slits or channels may keep the tubes and/or wires parallel (Recall, e.g., FIG. 7A.), and/or may spread the tubes and/or wires (Recall, e.g., the bottom of FIG. 7B and FIG. 7D.), and/or may concentrate the tubes and/or wires (Recall, e.g., the top of FIG. 7B and FIG. 7C.).

In one or more example embodiments, the surgical tool organizer is a foam material such as polyurethane foam, high density neoprene sponge foam, vinyl nitrate foam, vinyl nitrile foam, and the like. The properties of the foam material, such as density, firmness, support factor, tensile strength, resilience, and the like are such that medical instruments and/or supplies used during a medical procedure (such as an endoscopy procedure) are securely held within the slits or channels but can be rapidly and easily removed from and returned to the surgical tool organizer as needed. In some embodiments, at least part of the length of the tube(s) and/or wire(s) can be pulled (and/or pushed) through the slit or channel. The firmness and/or resilience of the foam material may be considered when determining the width of a channel relative to the diameter of the tube or wire to be held in the channel. For example, the width of a channel in a firmer foam might be very close (e.g., within a few percent of) the diameter of a tube or wire to be held, while the width of a channel in a softer foam might be significantly smaller (e.g., up to 50 percent less) than the diameter of a tube or wire to be held. Note that diameters of existing endoscope tubes and/or wires can be as small as 80-250 μm and up to 13 mm and more, and various diameters in between (e.g., 3 mm, 6 mm, 10 mm, etc.). However, the material of a channel and/or slit and/or the cross sectional dimensions of the channel and/or slit may be chosen to accommodate tubes and/or wires having other diameters.

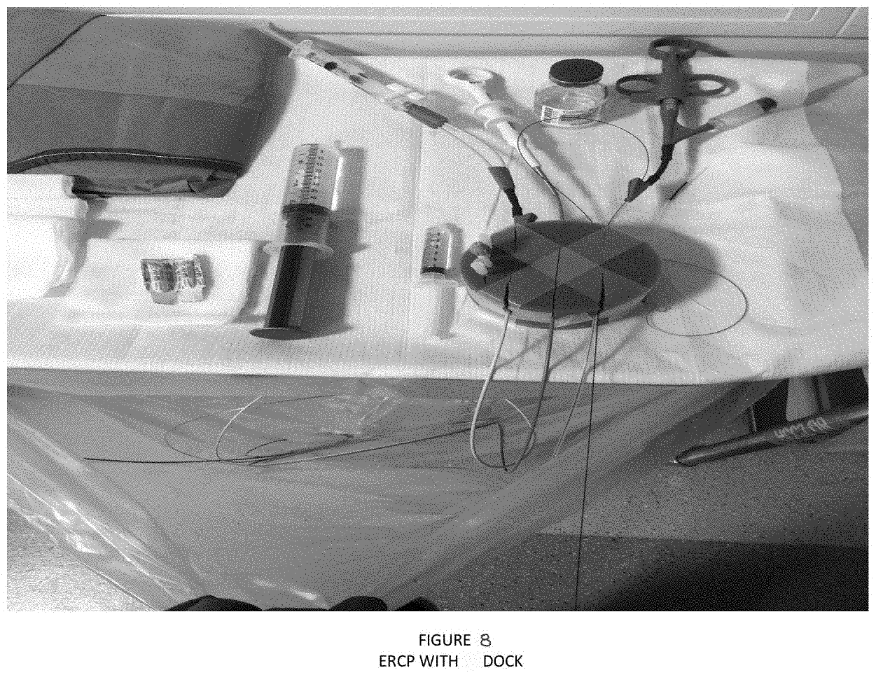

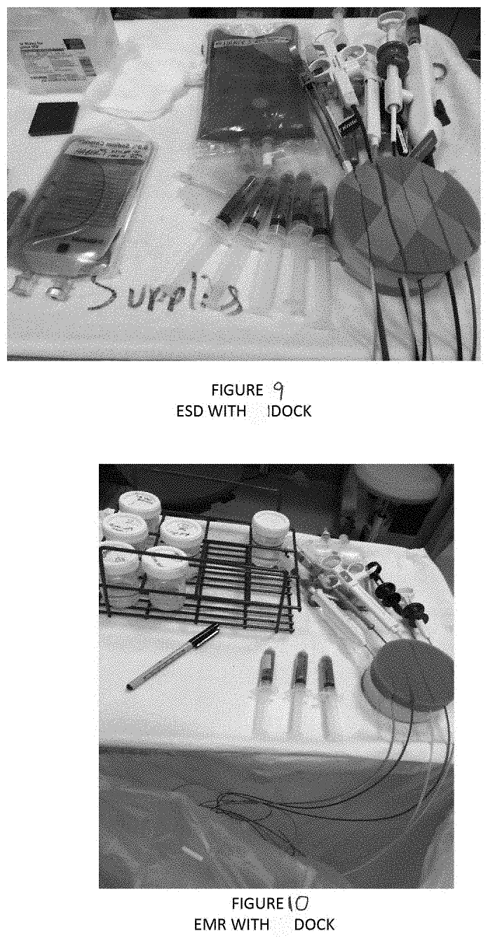

In some embodiments, the surgical tool organizer holding section has on the order of 4-10 slits having depths from 1 to 1.5 or 2 inches (Recall, e.g., FIG. 4.) and each on the order of 10 inches long. In various embodiments, the surgical tool organizer may be used to hold various medical equipment or supplies for endoscopy procedures (e.g., upper GI endoscopy procedures, lower GI endoscopy procedures, regular endoscopy procedures, advanced endoscopy produces such as endoscopic retrograde cholangiopancreatography (ERCP) (See, e.g., FIG. 8.), full-thickness resection (FTR), endoscopic submucosal dissection (ESD) (See, e.g., FIG. 9.), per oral endoscopic myotomy (POEM), gastrointestinal endoscopic mucosal resection (EMR) (See, e.g., FIG. 10.) and the like) such as endoscopes, duodenoscopes, full-thickness resection devices, CF-colonoscopes, 1T scopes, 2T scopes, Therapeutic scopes, Pediatric scopes, Xp scopes, Fuji Double Balloon scopes, Linear scopes, Radial scopes, and the like. For example, flexible portions of scopes (such as a cable, cord, tube, shaft, and the like) and/or tools that are (or that are to be, or that might be) inserted into an instrument channel of the endoscope and through the insertion tube of the endoscope may be placed within the various slits or channels of the holding section to keep the scopes organized in an orderly manner while easily accessible.

Within a single holding section, the slits or channels can have any combination of cross-sectional shapes and/or dimensions (Recall, e.g., FIGS. 6A-6E.), lengths, and/or paths (Recall, e.g., FIGS. 7A-7D.). These parameters should be chosen based on the size, flexibility, and/or length, etc., of the respective tube(s) and/or wire(s) to be used in the given medical procedure.

Although some embodiments were described as being made from foam, other materials having suitable characteristics may be used in addition, or instead.

Although some example embodiments were described as having a substantially flat upper surface, other example embodiments may have contoured (e.g., convex, concave, or some combination of convex and concave) upper surfaces.

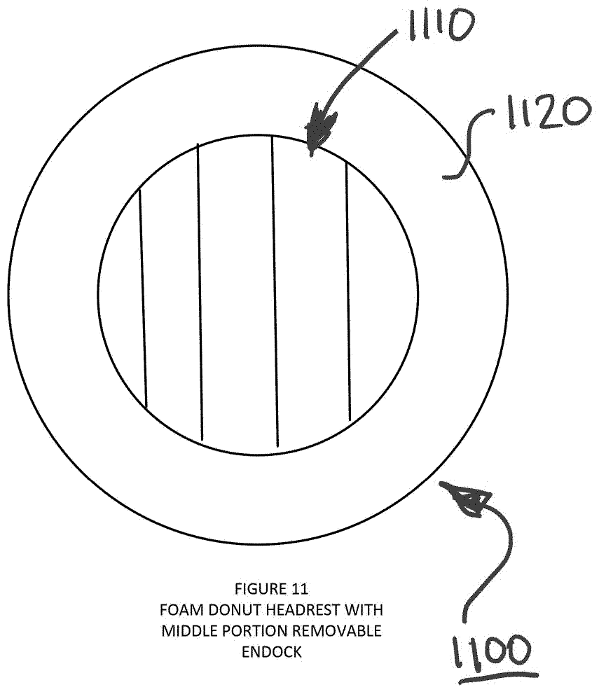

In certain embodiments, the surgical tool organizer may be part of a kit comprising one or more of the surgical tool organizer, a patient headrest, and a bag for use in retaining various items or portions of items used during the procedure. For example, referring to the plan view of FIG. 11, in some embodiments a kit 1100 may comprise a foam assembly having a separable center portion 1110 that will serve as the surgical tool organizer, where once the surgical tool organizer portion 1110 of the foam assembly is removed, the remaining portion 1120 of the foam assembly 1100 is suitably sized and shaped for use as a patient headrest. In one or more of such embodiments, the kit may further comprise one or more bags (e.g., a clear or substantially-clear bag), which may initially hold the foam assembly 1100 and which may be subsequently secured to the workspace (e.g., by one or more adhesive tabs) and utilized during a medical procedure to hold various items. (Recall, e.g., FIGS. 8 and 10.) In one example embodiment, the bag is approximately 20 inches wide and 37 inches high, but this is not limiting. Although the example of FIG. 11 includes a surgical tool organizer portion 1110 having parallel slits or channels, other path shapes can be used instead, or in addition. (Recall, e.g., FIGS. 7A-7D.)

### § 6.1 Example Methods of Use

Any of the foregoing docks can be used as follows. The dock is placed on a stand such as a mayo tray or horizonal surface of an endoscope tower. The dock has a bottom surface touching the stand and an upper surface having a plurality of slits or channels. Each of the plurality of slits or channels has an entry end and an exit end. The surgeon or their assistant then inserts at least some of the plurality of flexible tubes and/or wires into the dock so that each of the plurality of slits or channels grips a respective one of at least some of the flexible tubes and/or wires.

In example embodiments in which a bottom surface of the dock has covered adhesive, the method further includes removing the adhesive cover to expose the adhesive on the bottom surface of the foam component before the foam component is placed on the stand.

A bag may be attached or otherwise held to the stand. Then, at least a portion of the lengths of at least some of the flexible tubes and/or wires may be placed into the bag.

What is claimed is:

1. An endoscopic surgical tool organizer comprising:
a holder section having an upper surface, a substantially flat lower surface, and at least one peripheral edge,
wherein the upper surface has a plurality of slits or channels, each having an entry point and an exit point on the at least one peripheral edge, and each dimensioned to hold at least a portion of a length of an endoscopic insertion tube or endoscopic tool wire or tube, and
wherein the substantially flat lower surface is provided with a non-slip material, finish, or adhesive, and
wherein the holder section is dimensioned to fit on a tray of a mayo stand.

2. The endoscopic surgical tool organizer of claim 1, wherein the holder section is made from foam.

3. The endoscopic surgical tool organizer of claim 1, wherein the holder section is a foam block.

4. The endoscopic surgical tool organizer of claim 1, wherein the holder section is a foam disk.

5. The endoscopic surgical tool organizer of claim 1 wherein at least one of the plurality of slits or channels is a slit.

6. The endoscopic surgical tool organizer of claim 1 wherein at least one of the plurality of slits or channels is a channel having a U-shaped cross section.

7. The endoscopic surgical tool organizer of claim 1 wherein at least one of the plurality of slits or channels is a channel having a bulb-shaped cross section or a dovetail-shaped cross section.

8. The endoscopic surgical tool organizer of claim 1 wherein at least one of the plurality of slits or channels is a channel having a partially tapered cross section.

9. The endoscopic surgical tool organizer of claim 1 wherein at least two of the plurality of slits or channels have substantially parallel paths.

10. The endoscopic surgical tool organizer of claim 1 wherein at least two of the plurality of slits or channels have paths that spread out at an entry or exit side of the at least two of the plurality of slits or channels.

11. The endoscopic surgical tool organizer of claim 1 wherein at least two of the plurality of slits or channels have paths that concentrate at an entry or exit side of the at least two of the plurality of slits or channels.

12. The endoscopic surgical tool organizer of claim 1 wherein at least two of the plurality of slits or channels have paths that spread out at an entry or exit side of the at least two of the plurality of slits or channels, and that concentrate at an opposing exit or entry side of the at least two of the plurality of slits or channels.

13. The endoscopic surgical tool organizer of claim 1 wherein at least two of the plurality of slits or channels have paths that spread out at both an entry and an exit side of the at least two of the plurality of slits or channels.

14. A kit comprising:
a) a substantially ring-shaped head rest; and
b) a substantially circular dock, wherein the substantially circular dock is arranged within the head rest ring, is precut for easy separation of the substantially ring-shaped head rest and the substantially circular dock, and is provided with slits and/or channels sized to accommodate at least a portion of a length of a flexible tube used in an endoscopic surgical procedure.

15. The kit of claim 14 further comprising:
c) non-slip coating or adhesive provided on one side of the foam dock.

16. The kit of claim 15 wherein the non-slip coating or adhesive is not provided on the head rest.

17. The kit of claim 14 further comprising:
c) a bag to support and/or contain at least a portion of flexible tubes, wherein the bag is the same bag used to seal head rest and the dock.

18. The kit of claim 16 wherein the bag includes an extended tab at one end of the bag opening, wherein the extended tab is dimensioned to tuck under a mayo stand tray.

19. A method for organizing endoscopic equipment including a plurality of flexible tubes/wires, a (mayo) stand, the method comprising:
a) placing a foam component on the stand, wherein the foam component has a bottom surface touching the stand and an upper surface having a plurality of slits or channels, each of the plurality of slits or channels having an entry end and an exit end; and
b) inserting at least some of the plurality of flexible tubes/wires into the foam component so that each of the plurality of slits or channels grips a respective one of at least some of the flexible tubes/wires.

20. The method of claim 19 wherein the bottom surface of the foam component has covered adhesive, the method further comprising:
removing the cover to expose the adhesive on the bottom surface of the foam component before the foam component is placed on the stand;
attaching a bag to the stand; and
placing at least a portion of the lengths of at least some of the flexible tubes/wires into the bag.
